(19) United States
(12) Patent Application Publication
(10) Pub. No.: US 2016/0242965 A1
(43) Pub. Date: Aug. 25, 2016

(54) Aerated materials

(71) Applicant: ONBONE OY, Helsinki (FI)

(72) Inventor: Antti Pärssinen, Helsinki (FI)

(21) Appl. No.: 15/030,871

(22) PCT Filed: Oct. 21, 2014
(86) PCT No.: PCT/FI2014/050793

§ 371 Date: Apr. 21, 2016

(30) Foreign Application Priority Data
Oct. 21, 2013 (FI) 20136039

(51) Int. Cl. A61F 13/04; C08K 5/00; A61F 5/058

(52) U.S. Cl. CPC A61F 13/04; A61F 5/058; C08K 5/00; A61F 2013/00225

(57) ABSTRACT

Aerated composite materials in the shape of a three-dimensional objects and methods of producing the same. The composite comprises a thermoplastic biodegradable polyester mixed with reinforcing agents selected from particles of woody materials having a smallest dimension greater than 0.1 mm. The composite material further comprises regions of elasticity formed by incisions which upon stretching will yield open pores. The incised regions will also provide for flexibility or semi-rigidity in at least one dimension. The material can be used in comfortable splints and circumferential casts.

### TECHNICAL FIELD

The present invention relates to wood-plastic composite materials. In particular, the present invention concerns composite materials comprising a thermoplastic polymer and a reinforcing component, which composite materials exhibit mechanical properties in the range from flexible to semi-rigid. Methods of producing such materials as well as uses of the materials are also disclosed.

### BACKGROUND ART

Casting is the most common form of external splinting and it is used for a wide array of bone and soft-tissue injuries. In this context, the function of the cast is to immobilize and to protect the injury and, especially, to minimize motion across a fracture site.

A number of casting materials are known. The first generation of casting material is formed by plaster-of-Paris (in the following abbreviated “POP”). Largely owing to its low cost and ease of molding it has gained universal acceptance. There are, however, a number of disadvantages of POP, including long setting times, messy application, low strength and relative heaviness. Although setting takes only a few minutes, drying may take many hours or days, especially if the atmosphere is moist and cool. Impacts on the plaster while it is setting may cause a weakening of the material. Furthermore, the transparency to X-rays (in the following “radiolucency”) is poor.

The second generation of casting materials is formed by synthetic composite materials, such as fiberglass reinforced polyurethane resins. They are useful alternatives to conventional plaster-of-Paris and are gaining increasing popularity. Fiberglass and resinous materials can safely be applied as external splints. These materials are lightweight, durable and waterproof but require protective packaging and some indications are difficult to apply. Further on, some of the fiberglass casting materials during applying requires special gloves for avoiding penetration of small fiberglass particles through skin. In addition, synthetic casting materials may have a shorter setting and solidification time than traditional plaster-based materials. Further, they are much more expensive than plaster at present, but to balance this disadvantage, fewer bandages are required and they are much more durable in everyday use. They are also more radiolucent than plaster based casting materials.

Further developments in the field of casting techniques have included the idea of the application of semi-rigid and rigid materials to a cast (focused rigidity), as that enables controlled functional load and stress structures in casts which thus improved restoration of the function of the affected limb. As an evidence of this casting technique Softcast (3M product) is widely used material in fracture treatment.

In addition to the above mentioned synthetic toxic casting materials, various composites consisting of polycaprolactone homopolymers (PCLs) and fibrous materials are known. Examples of such materials can be found in WO 2006/027763A2, WO 2007/035875, US 2008/0262400 and US 2012/0071590. Some of the materials have orthopedic applications.

WO 94/03211 discloses a composite of cellulosic filler and polycaprolactone having improved moisture-permeability. WO 2006/027763A2 discloses geometrically apertured splint manufactured from PCL and a lignocellulose filler. WO 2007/035875 discloses a cross-linked thermoplastic material with aramide fibers, into which some wood pulp or natural fibers has been incorporated. US Patent Application No. 2008/0103423 concerns a combination of cork and polycaprolactone which exhibits some degree of flexibility. Published Patent Application US 2012/0071590 describes composite material comprising hard wood chips and high molecular weight polycaprolactone useful for fracture management of upper and lower limbs.

### SUMMARY OF INVENTION

### Technical Problem

For some orthopedic immobilization applications it is required that a body support or parts of it are flexible or elastic. This is the case especially in large splints and in circumferential casts. The known PCL based thermoplastic composite materials are rigid, and do not allow freedom of movements and swelling of the limbs in orthopedic applications.

There is a need for materials which, while exhibiting the advantageous properties of thermoplastic/wood particle based composites, also have sufficient flexibility for use in semi-rigid immobilization of treated bony premises.

It is an aim of the present invention to provide novel composite materials in the shape of three-dimensional objects exhibiting at least in one dimension mechanical properties extending from flexible to semi-rigid.

It is another aim of the present invention to provide methods of producing such materials.

It is still a further aim of the present invention to provide for the use of the novel flexible or semi-rigid materials.

### Solution to Problem

The present invention is based on the concept of providing composite materials, which are moldable and workable at temperatures below 70° C., by combining a first component formed by a rigid thermoplastic polymer, a second component formed by a reinforcing material and introducing into the composite materials at least one region of non-rigidity to provide for objects having properties of flexibility or semi-rigidity in at least one dimension.

Compositions of the indicated kind can be produced by incorporating into the composite materials perforation for example in the form of incisions, in particular unidirectional incisions, to achieve properties of increased flexibility or semi-rigidity.

The novel materials can be shaped into blanks or bandages or other three-dimensional products or objects which can be used for orthopedic immobilization wherein the body support or parts of it are flexible or elastic.

Examples of suitable application include splints and circumferential casts.

More specifically, the present composite products are characterized by what is stated in the characterizing part of claim 1.

The method according to the present invention is characterized by what is stated in the characterizing part of claim 27.

The novel use according to the present invention is characterized by what is stated in the characterizing part of claim 32.

### Advantageous Effects of Invention

The materials of the present invention can be heated to working temperature at which the composition can be easily formed by hand to various 3D shapes for example to contour the human anatomy. The composition solidifies upon cooling. A semi-rigid, typically at least partially flexible or elastic structure is obtained which can be readily achieved without chemically modifying the composition of the material. Thus, by mechanically processing the primarily rigid material it can be made semi-rigid or flexible in at least one dimension.

The materials can be used for therapy and for sports applications. They allow small movements of an immobilized limb or body part. User or patient comfort is greatly increased compared to conventional rigid splints or circumferential casts, in particular since the incisions upon stretching will yield apertures which increases breathability of the material.

These materials can also be combined easily with the similar rigid compositions allowing the clinician to choose the level of rigidity necessary to injury site.

The composition according to invention can be re-heated an unlimited number of times after the form has settled. In other words, formability is reversible without the composition being damaged.

Next, embodiments will be examined with reference to the attached drawings.

### BRIEF DESCRIPTION OF DRAWINGS

FIG. 1 is a schematic presentation of one possible incision pattern applied to a rectangular blank formed by the present material;

FIG. 2 The compression test set up with WOODCAST test specimen being tested;

FIG. 3A is a photograph of incised WOODCAST® 2 mm sample stretched 15%;

FIG. 3B is a photograph of incised WOODCAST® 2 mm sample stretched 30%;

FIG. 4 shows photographs of incised WOODCAST® test specimens, stretched at various stretching ratios;

FIG. 5 shows the effect of stretching on the pore area of incised composite materials; and

FIG. 6 is a schematic presentation of the opening process of incision.

FIG. 7 illustrates the cutting patterns when preparing Ring Stiffness specimens for Examples 1 and 2 (Table 1).

### DESCRIPTION OF EMBODIMENTS

In the present context, the term “moldability” means that a composition can be heated to working temperatures at which the composition can be easily formed by hand to various 3D shapes, for example to contour the human anatomy, and the composition solidifies to a semi-rigid structure upon cooling.

“Rigid” when used in the context of a polymer means that the polymer is essentially non-flexible under conventional forces exerted by a person's limb or body part immobilized by the material for example in the shape of a splints or circumferential casts. “Semi-rigid” by contrast means that the composite material, under the same conditions and forces, allows from some movement in at least one direction.

“Biodegradable” typically stands for materials capable of undergoing decomposition into carbon dioxide, methane water, inorganic compounds, or biomass in which the predominant mechanisms is the enzymatic action of micro-organisms that can be measured by standard tests, over a specific period of time.

“Ambient temperature” stands for a temperature of about 10 to 30° C., in particular about 15 to 25° C.

A property of “flexibility” can be measured by a ring stiffness test, and such a property will be manifested in a greatly reduced stiffness. Typically the stiffness will be at least 20% lower, preferably at least 30% lower than for a corresponding material without incisions or other perforations.

As discussed above, a novel composite material according to the present technology comprises generally a material which is shaped into a three-dimensional object.

“Blank” stands for a piece of the present composite material which can, if so desired, be processed into a shape more suitable for use as an orthopedic device by mechanically processing, for example by cutting if needed, and typically by molding against a body part to be immobilized at a temperature above the softening point of the material. Typically, a “blank” is planar.

Within the scope of the present technology, examples of three-dimensional objects include planar structures having two opposite at least essentially planar surfaces. Such structures have a length of typically 10 to 2500 mm, a width or breadth of typically 5 to 1000 mm and a thickness of typically 0.1 to 100 mm. The structure can also be a bandage or tape which for example has a length of 0.5-10 m, thickness of 0.5-1.5 mm and width of 2.5-15 cm.

In addition to planar structures, also other three-dimensional structures are possible, such as cylindrically and conically or even spherical objects as well as various chute-shaped objects, each of these optionally having one or more bent portions.

The below embodiments relate to composite materials shaped into generally elongated, planar structures (i.e. “blanks”) which subsequently can be formed, e.g. into a tubular shape or as a chute. As will be discussed, such planar structures exhibit increased flexibility or softness in transversal direction, i.e. perpendicular to the longitudinal axis of the plane. That property will be retained during the shaping of the planar structure.

Basically, the present composite material comprises a first component formed by a polymer and a second component formed by a reinforcing material. The first component comprises typically a thermoplastic polymer selected from the group of biodegradable polyesters and mixtures thereof. The second component comprises particles of a woody material, having a smallest dimension greater than 0.1 mm.

According to an aspect of the present invention, the first component forms the matrix of the composite.

The microstructure of the second component in the composition is discontinuous. Preferably the second component is embedded into the matrix formed by the first component.

The particles of the second component can have random orientation or they can be arranged in a desired orientation. The desired orientation may be a predetermined orientation.

According to a preferred embodiment, a polycaprolactone polymer (in the following also abbreviated “PCL”) is used as a thermoplastic polymer in the first component of the composition. The polycaprolactone polymer is formed by repeating units derived from epsilon caprolactone monomers. The polymer may be a copolymer containing repeating units derived from other monomers, such as lactic acid, glycolic acid, but preferably the polymer contains at least 80% by volume of epsilon caprolactone monomers, in particular at least 90% by volume and in particular about 95 to 100% epsilon caprolactone monomers.

In a preferred embodiment, the thermoplastic polymer is selected from the group of epsilon-caprolactone homopolymers, blends of epsilon-caprolactone homopolymers and other biodegradable thermoplastic homopolymers, with 5-99 wt %, in particular 40 to 99 wt %, of an epsilon-caprolactone homopolymer and 1-95 wt %, in particular 1 to 60 wt %, of a biodegradable thermoplastic polymer, and copolymers or block-copolymers of epsilon-caprolactone homopolymer and any thermoplastic biodegradable polymer, with 5 to 99 wt %, in particular 40 to 99 wt % of repeating units derived from epsilon-caprolactone and 1 to 95 wt-%, in particular 1 to 60 wt-%, repeating units derived from other polymerizable material.

Examples of other biodegradable thermoplastic polymers include polylactides, poly(lactic acid), polyglycolides as well as copolymers of lactic acid and glycolic acid.

The first polymer component, in particular the epsilon caprolactone homo- or copolymer, has an average molecular weight of 60,000 to 500,000 g/mol, for example 65,000 to 300,000/mol, in particular at least 80,000 g/mol, preferably higher than 80,000 and up to 250,000.

The molding properties of the present invention can be determined by the average molecular weight (Mn) of the polymer, such as epsilon caprolactone homo- or copolymer. A particularly preferred molecular weight range for the Mn value of PCL is from about 100,000 to about 200,000 g/mol. The number average molar mass (Mn) and the weight average molar mass (Mw) as well as the polydispersity (PDI) were measured by gel permeation chromatography. Samples for GPC measurements were taken directly from the polymerization reactor and dissolved in tetrahydrofuran (THF). The GPC was equipped with a Waters column set styragel HR (1, 2 and 4) and a Waters 2410 Refractive Index Detector. THF was used as eluent with a flow rate of 0.80 ml/min at a column temperature of 35° C. A conventional polystyrene calibration was used. In determination of the water content of the monomer at different temperatures a Metroohm 756 KF Coulo meter was used.

The properties of moldability of the present composition can also be determined by the viscosity value of the polymer. For an epsilon caprolactone homopolymer: when the inherent viscosity (IV)-value of PCL is less than 1 dl/g the composite is sticky, flows while formed and forms undesired wrinkles while cooling. When PCL having IV-value closer to 2 dl/g is used the composite maintains its geometry during molding on the patient and it may be handled without adhesive properties. Thus, IV values in excess of 1 dl/g are preferred, values in excess to 1.2 dl/g are preferred and values in excess of 1.3 dl/g are particularly suitable. Advantageously the values are in the range of about 1.5 to 2.5 dl/g, for example 1.6 to 2.1 dl/g. Inherent Viscosity values were determined by LAUDA PVS 2.55 d rheometer at 25° C. The samples were prepared by solvating 1 mg of PCL in 1 ml chloroform (CH₃Cl).

A particularly important feature of the thermoplastic polymer is the viscosity which is relatively high, typically at least 1,800 Pas at 70° C., 1/10 s; the present examples show that the viscosity can be on the order of 8,000 to 13,000 Pas at 70° C., 1/10 s (dynamic viscosity, measured from melt phase). Below the indicated value, a reinforced material readily wrinkles during forming it on a patient.

The thermoplastic material is preferably a biodegradable polymer (only) but also non-biodegradable polymers may be utilized. Examples of such polymers include polyolefins, e.g. polyethylene, polypropylene, and polyesters, e.g. poly(ethylene terephthalate) and poly(butylenes terephthalate) and polyamides. The polymer may also be any cross-linked polymers manufactured prior to processing or in situ during the compounding process for example by means of ionizing radiation or chemical free-radical generators. Examples of such polymers are cross-linked polyesters, such as polycaprolactone.

Combinations of the above biodegradable polymers and said non-biodegradable polymers can also be used. Generally, the weight ratio of biodegradable polymer to any non-biodegradable polymer is 100:1 to 1:100, preferably 50:50 to 100:1 and in particular 75:25 to 100:1. Preferably, the composite material has biodegradable properties greater, the material biodegrades quicker or more completely, than the thermoplastic material alone.

By using an additional polymer component in the polymer material of the first component, mechanical properties of the first component can be improved. Such mechanical properties include tear-resistance.

According to the invention, a polymer of the afore-said kind is preferably moldable at a temperature as low as +58° C., in particular at +65° C. or slightly above, and it can be mixed with wood particles or generally any porous material gaining increased rigidity of the formed composite. The polymer component, such as polycaprolactone homopolymer, defines the form of the splinting material against the skin.

In one embodiment, the first polymer component has a melt flow index of about 0.3-2.3 g/min (at 80° C.; 2.16 kg).

The composite material according to the present technology typically exhibits formability at a temperature of about 50 to 70° C. and it is rigid at a temperature of less than 50° C., in particular at ambient temperature up to at least 45° C.

The second component is a reinforcing material which comprises or consists essentially of a woody material having a smallest diameter of greater than 0.1 mm. There can also be other wood particles present in the second component. The woody material can be granular or platy. According to one embodiment, the second component comprises a woody material derived from platy wood particles having a smallest diameter of greater than 0.1 mm. Thus, generally, the wood component can be characterized generally as being greater in size than powder.

The size and the shape of the wood particles may be regular or irregular. Typically, the particles have an average size (of the smallest dimension) in excess of 0.1 mm, advantageously in excess of 0.4 mm, for example in excess of 0.5 mm, suitably about 0.6 to 10 mm. The length of the particles (longest dimension of the particles) can vary from a value of greater than 0.6 mm to value of about 1.8 to 200 mm, for example 3 to 21 mm. The woody particles can be granular, platy or a mixture of both. Woody particles considered to be granular have a cubic shape whose ratio of general dimensions are on the order of thickness:width:length=1:1:1. In practice it is difficult to measure each individual particle to determine if it is a perfect cube. Therefore, in practice, particles considered to be granular are those where one dimension is not substantially different from the other two.

Woody particles considered to be platy means that they have generally a plate-shaped character, although particles of other forms are often included in the material. The ratio of the thickness of the plate to the smaller of the width or length of the plate's edges is generally 1:1 to 1:500, in particular about 1:2 to 1:50. Preferably, the woody particles include at least 10% by weight of chip-like particles, in which the ratio of general dimension are on the order of thickness:width:length=1:1-20:1-100, with at least one of the dimension being substantially different than another.

Based on the above, the platy particles of the present invention generally comprise wood particles having at least two dimensions greater than 1 mm and one greater than 0.1 mm, the average volume of the wood particles being generally at least 0.1 mm³ more specifically at least 1 mm³.

“Derived from platy wood particles” designates that the wood particles may have undergone some modification during the processing of the composition. For example, if blending of the first and second components is carried out with a mechanical melt-mixing device or with extruder having small nozzle dimensions, some of the original platy wood particles may be deformed to an extent.

The majority of wood particles greater in size than powder, which particles may be granular or platy, typically make up more than 70% of the woody material. The wood species can be freely selected from deciduous and coniferous wood species alike: beech, birch, alder, aspen, poplar, oak, cedar, Eucalyptus, mixed tropical hardwood, pine, spruce and larch tree for example. Other suitable raw-materials can be used, and the woody material of the composite can also be any manufactured wood product.

The particles can be derived from wood raw-material typically by cutting or chipping of the raw-material. Wood chips of deciduous or coniferous wood species are preferred, such as chips of aspen or birch.

In addition to wood chips and other platy particles, the present composition can contain reinforcing fibrous material, for example cellulose fibers, such as flax or seed fibers of cotton, wood skin, leaf or bark fibers of jute, hemp, soybean, banana or coconut, stalk fibers (straws) of hey, rice, barley and other crops and plants including plants having hollow stem which belong to main class of Tracheobionta and e.g. the subclass of meadow grasses (bamboo, reed, scouring rush, wild angelica and grass).

The composition may contain particulate or powdered material, such as sawdust, typically having particles with a size of less than 0.5 mm*0.5 mm*0.5 mm. Particulate or powdered material is characterised typically as material of a size in which the naked eye can no longer distinguish unique sides of the particle. Platy particles are easily recognizable as one dimension is recognizable by the naked eye as being larger than another. Granular particles, while having substantially equal dimensions, are of such dimension that their unique sides can be determined by the naked eye and oriented.

In one embodiment, the woody material comprises platy wood particles or particles obtained from such wood particles, by crushing, said particles forming about 30 to 100% of the total weight of the second component.

The compounding of the first and the second component is typically carried out in, e.g., an extruder, in particular a single or dual screw extruder. In the compounding process the screw extruder profile of the screw is preferably such that its dimensions will allow relatively large wood chips to move along the screw without crushing them. Thus, the channel width and flight depth are selected so that the formation of excessive local pressure increases, potentially causing crushing of the wood particles, are avoided. The temperature of the cylinder and the screw rotation speed are also selected such as to avoid decomposition of wood chip structure by excessively high pressure during extrusion. For example a suitable barrel temperature can be in the range of about 110 to 150° C. from hopper to die, while the screw rotation speed was between 25-50 rpm. These are, naturally, only indicative data and the exact settings will depend on the actual apparatus used.

A composition comprising merely the first and the second components typically is rigid. The polymer of the first component is hard.

This kind of composition is, according to the present technology, converted to a semi-rigid structure with help of mechanical processing.

According to the invention, a semi-rigid composite is achieved by providing punctuation of the composite, for example with unidirectional incisions. The size, frequency and perforation pattern may vary.

In an embodiment of invention, elastic or soft polymer as third component and punctuation are used to enhance the overall flexibility of a orthopedic support according to invention. This embodiment is disclosed in more detail in our co-pending patent application titled “Novel materials” filed on 21 Oct. 2013.

The composite material manufactured of only polycaprolactone (PCL) polymer and wood chips has limited capability to resist tearing during use in the application temperature of ˜65° C. Therefore, the design of aerating holes must be carefully selected to avoid additional weakening of the tear strength of the composite material in use. In addition, the composite material is used for immobilization of limbs of the human body, therefore formation of weak spots, caused by holes in material, must be avoided.

All the traditional readily open hole shapes and patterns are not feasible for the present materials because there is no way of controlling the level of openings in the areas which require maximum strength. By using the present method, which will be discussed in more detail below, controlled perforation can be achieved by first forming incisions into the composite material and by then applying directional finishing, excellent mechanical strength also in the weak spots can be obtained.

By introducing lengthwise incisions in the composite profile, aerating can be achieved by merely widening the material in widthwise direction. Such widening of the material will typically take place during treatment of injured body extremities with the present materials.

The present incisions are located such that they are kept in “closed” status in the areas of the orthopaedic device requiring maximal strength so as not to impair mechanical strength.

Typically the areas requiring maximal strength are subjected to longitudinal forces, i.e. forces which act along the length of the device. Thus, in one preferred embodiment, the incisions are longitudinally directed, and they will therefore not be opened by the action of such longitudinal force.

In short, in one embodiment, immobilization using the present composite materials, shaped as three-dimensional objects, requires that the orthopaedic device is rigid in the longitudinal direction of the object. By orientating the incisions longitudinally, the incisions will remain closed under the influence of longitudinal forces, and the material will exhibit mechanical strength and rigidity directly derivable from the structure of the material. No elasticity will be needed in the longitudinal direction, or indeed even desired.

By contrast, immobilization will typically not require stiffness and rigidity in the transversal direction, and the orthopaedic device will not be subjected to transversal forces due to immobilization. However, user comfort may require some flexibility of the material, to allow for some movement, and the present materials will therefore yield to forces perpendicular to the general orientation of the incisions by opening the close incisions. The incisions may even by fully opened always close to the circular shape.

In the context of the present technology, the pattern and the shape of the incisions in the composite material have been studied in particular for planar composite materials having a thickness in the range of 2 to 4 mm.

The incisions studied are formed by straight (linear) incisions or cuts. Preferably there are lines formed with consecutive incisions. In particular there is a plurality of such lines, which preferably are parallel.

In a particular embodiment, laterally to the linear direction of the incisions, incisions are phased off. Thus, two adjacent incisions are not located along the same transversal line.

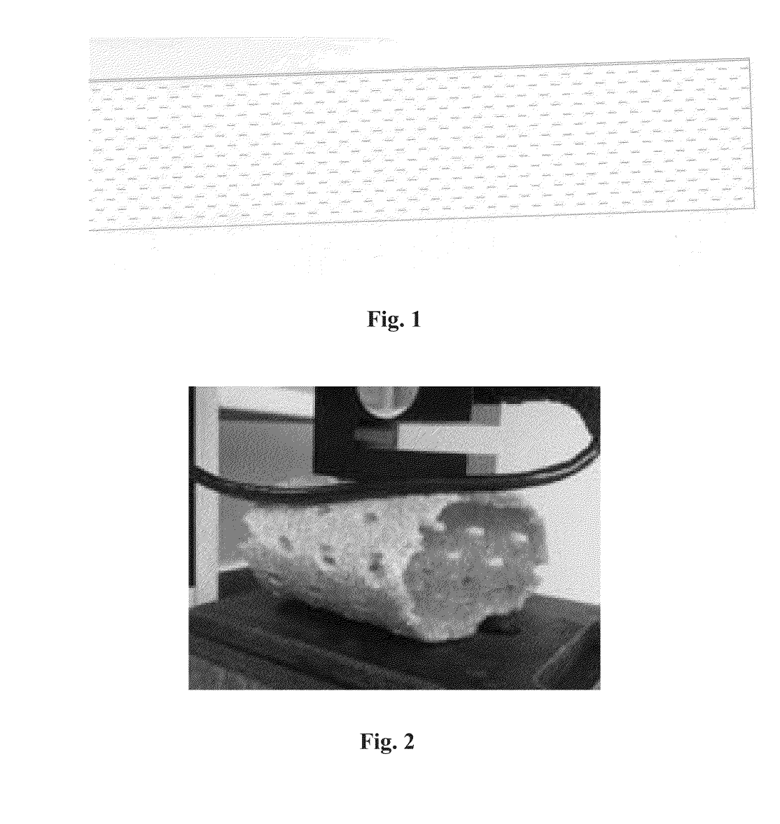

This is shown in FIG. 1, which depicts a composite material comprises a plurality of perforations in the form of linear incisions which are all unidirectionally orientated. As can be seen, the incisions are arranged in rows to form a plurality of parallel linear lines. Incisions in adjacent rows are never located along the same transversal line drawn through the center of the incisions.

The incision lines together form a region of flexibility in the mechanically processed material. When subjected to the stretching laterally, the incisions will form apertures (FIG. 6). The figure shows the effect of latitudinal stretching on a longitudinally incised composite material.

Typically, the object will be stretched at least 5%, typically up to 75%, in particular about 10 to 50%.

Subject to stretching the novel aerated material will have pore area which is 2× to 100×, typically 2.5× to 15× greater that than pore area of the corresponding non-stretched, non-incised article (object). The pore area can be about 2.5 to 30% of the total area, for example about 3 to 20%, for example about 5 to 15%.

It has been found that incisions having a length of generally more 20 mm may cause tearing of the material when exposed to strong twisting and strain. On the other hand, incisions which are less than 5 mm in length do not sufficiently open during applying the material on human limb to allow for proper aerating.

Further, the space between each incision in longitudinal direction must exceed 5 mm to avoid tearing of the material and be less than 20 mm to achieve sufficient level of aerating.

The space between each incision line transversally to the linear incision must exceed 10 mm to avoid tearing and be less than 25 mm to achieve sufficient level of aerating.

The incisions may be manufactured into the composite profile with an incision device, examples of suitable equipment include a rolling cylinder or a press equipped with blades, water jet, and laser cutting.

Typically, the incisions have a width of 0.1 to 1 mm, preferably 0.3 to 0.8 mm, and a length of 4 to 20 mm.

The incisions can be made with a blade, the surface area of which incisions being on the blade ingoing side about 1 to 10 mm², preferably 2.5 to 8 mm².

The composite material according to any of the preceding claims, comprising incisions, the amount of the incisions/10 cm² being generally 20 to 100, preferably 30 to 70.

Stretching the material at application temperature, the practician or clinician may adjust the flexibility of a set product by controlling the aperture openings.

The particular advantage of incorporating incisions into the material is that upon stretching when the material is applied on a patient, the incisions will yield openings which give the material properties of breathability. Thereby skin maceration can be avoided.

The shape of the openings or apertures formed by stretching of the incision can be, for example, round, rectangular, square, diamond, hexagonal, oval, slot or ornamental perforation. The surface area of one hole should be generally about 3 to 30 mm² and amount of the holes is kept between 20 holes/10 cm² and 100 holes/10 cm². The total open area is less than 10 percentage of the whole surface area.

Manufacturing of an adhesive composite material comprising of only adhesive thermoplastic polymer and wood component by extrusion is a straightforward process. Openings of the indicated kind can also be formed into the material during or subsequent to the extrusion process.

The following non-limiting examples illustrate the invention:

### EXAMPLE 1

Ring Stiffness

The principles of standard ISO 9699:2007 were followed in the test set up. The ring stiffness was measured by recording the force and the deflection while compressing the cylinder at a constant deflection speed at vertical direction as seen in FIG. 1. Cross head speed of 20 mm/min was used in this test and the deflection was carried on until 50% deflection of the diameter of the cylinder was achieved. A plot of force versus deflection was generated using materials testing machine (LLOYD LR30K, Lloyd instruments, Southampton, UK) with 1 kN load cell for each specimen. In each series six or four samples were tested. The ring stiffness was calculated as a function of the force necessary to produce a 3% diametric deflection to the ring.

The ring stiffness values of cylinders were calculated by using the following equation (ISO 9699:2007):

S=(0.0186+0.025(y/dᵢ)) (F/Ly)×10⁶

where:
F=the force, that corresponds to a 3.0% deflection;
L=length of the test piece;
dᵢ=inner diameter of the ring;
y=the deflection, that corresponds to a 3.0% deflection.

For each series averages and standard deviations were calculated.

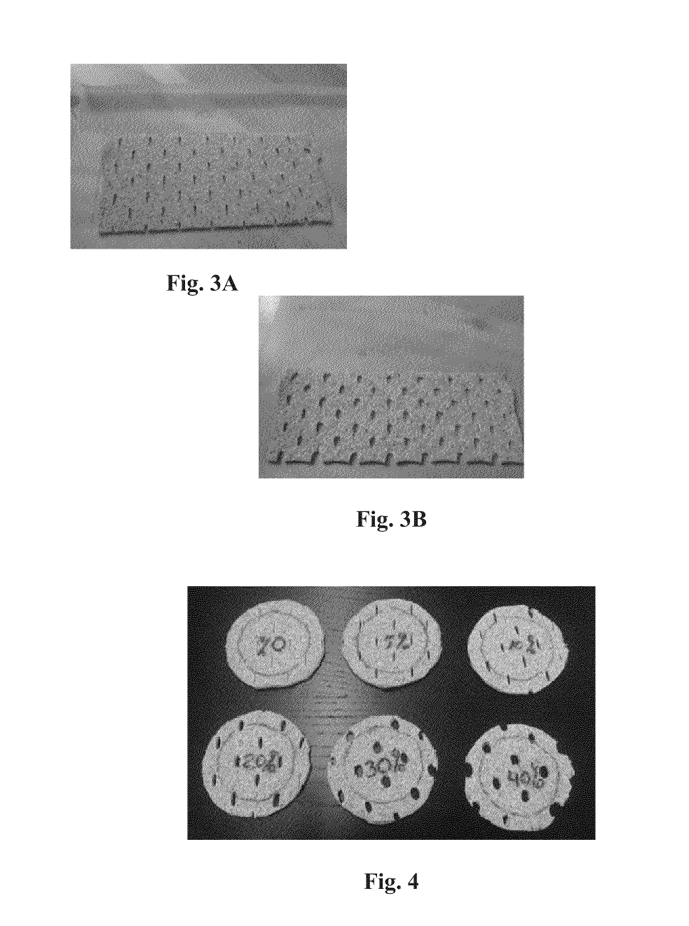

Some of the incised WOODCAST Splint and WOODCAST 2 mm samples were stretched either 15% or 30%. The stretched incised WOODCAST 2 mm samples are shown in FIGS. 2 and 3.

A series of cylindrical test specimen with diameter of app. 75 mm and length of 80 mm were manufactured according to instructions from manufacturer. With Scotchcast™ material samples with three, four and five layers of tape were manufactured, as there are considered to be the clinically most commonly used structures⁵⁻⁶. With WOODCAST products only one material layer was used. Diameter and length of each cylinder was measured with calibre (average of three measurements was used with each specimen).

The average ring stiffness values as well as standard deviations are shown in Table 1.

TABLE 1
Details of the materials tested and average ring stiffness values.

| | | | Ring stiffness | |
|---|---|---|---|---|
| | | | (kN/m²) | |
| | | | average and | |
| Trade Name | N | Details of the sample | standard deviations | |
| WOODCAST ® | 6 | Original | 154.4 | (+/−28.1) |
| Splint | | | | |
| WOODCAST ® | 4 | Incised only - no stretching | 79.9 | (+/−6.9) |
| Splint incised | 4 | Incised - 15% stretched | 71.0 | (+/−8.3) |
| | 4 | Incised - 30% stretched | 52.2 | (+/−5.7) |
| | 4 | Incised - 30% str. | 76.5 | (+/−2.4) |
| | | perpendicular | | |
| WOODCAST ® | 6 | Original | 33.0 | (+/−1.2) |
| 2 mm | | | | |
| WOODCAST ® | 4 | Incised only - no stretching | 22.1 | (+/−1.4) |
| 2 mm incised | 4 | Incised - 15% stretched | 17.7 | (+/−1.2) |
| | 4 | Incised - 30% stretched | 13.1 | (+/−6) |
| WOODCAST ® | 6 | 1 layer of material | 33.0 | (+/−1.2) |
| 2 mm | | | | |
| Scotchcast ™ | 6 | 5 layers of material | 175.2 | (+/−42.6) |
| | 6 | 4 layers of material | 80.8 | (+/−4.4) |
| | 6 | 3 layers of material | 41.1 | (+/−5.6) |

As seen in Table 1 there is large variation in stiffness properties of the materials. The WOODCAST 4 mm splint, Scotchcast™ with 5 layers of tape have similarly high stiffness values. Three layers of Scotchcast™ 3L and WOODCAST 2 mm Splint have very similar force versus elongation profile (not shown), though the stiffness of Scotchcast™ 3L is slightly higher. The stiffness of incised, stretched WOODCAST Splint samples is at same level with 4 layers of Scotchcast™ material. As can seen in Table 1 incisions greatly affects the stiffness of material, as expected. Stretching of sample further decreases the strength. The direction of load also greatly affects the stiffness (See values of incised WOODCAST Splint samples stretched 30%, when test rings are manufactured incisions being either parallel or perpendicular in the cylinder axis.)

All the test samples except Scotchcast™ L5 and Incised and stretched WOODCAST Splint were able to resist the compression at the test range, up to 50% of the diameter of test cylinders without fractures in materials. However with Scotchcast™ L5 and WOODCAST Splint samples structural fractures occurred only after approximately at 33% deformation, so it does not have any clinical relevance. The stiffness was proportional to the number of layers used with Scotchcast™ and WOODCAST Ribbon materials, as expected.

The circular shape of WOODCAST Ribbon, WOODCAST 2 mm, WOODCAST Splint samples and Scotchcast™ L3 and L4 mostly recovered, and no visual damage was observed in the tested samples. In none of the tested materials any signs of delamination between the layers were observed.

### EXAMPLE 3

Incised composite samples were either used as native, or stretched to enable opening of the voids in structure. The stretching ratios of the samples were as follows; 0%, 5%, 10%, 20%, 30%, and 40%. In FIG. 4 the actual test specimens are shown.

The total area of the voids in samples was first measured by first copying the samples in copying machine. From the copied papers the 2D-pictures of samples the weight of whole sample areas was first measured. The void areas were then cut out from the paper copies and the weights of these samples (with void areas removed) were then measured. This test was repeated three times with each of the samples, and the average void area was then calculated for each sample type.

To perform the vapour permeability analysis a thermo gravimetric analysed, HR73 (Mettler Toledo, USA) was used. The constant temperature of 50° C. was used with all the samples through the tests. From each sample type a circular sample with diameter approximately 60 mm was cut. 2 ml of distilled water was laced to a petri dish and the sample was placed over the dish. The sample was further sealed with aluminium tape from the sides and the edges of sample. With each sample care was taken that the open non-sealed area was constant to enable accurate comparative results. The petri dish was then placed into analyser and the weight change was recorded over 60 minutes with 5 minutes time intervals. From the obtained data the vapour permeation rate was then calculated.

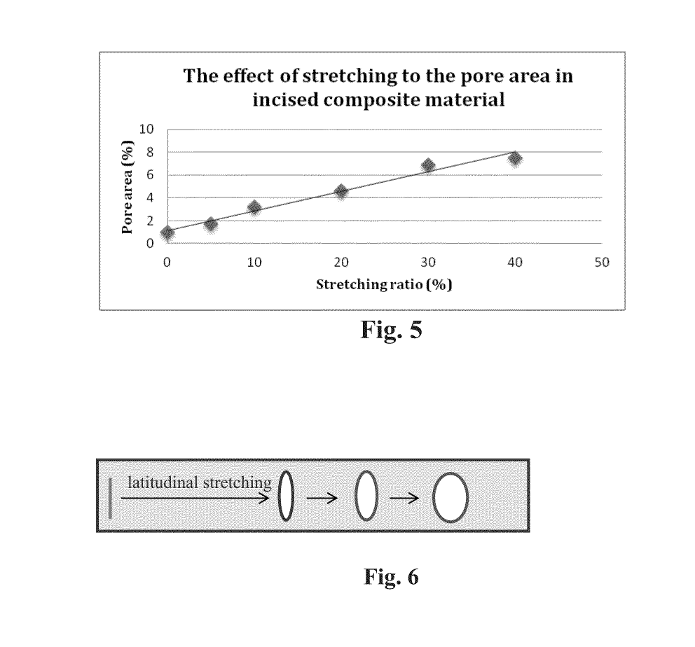

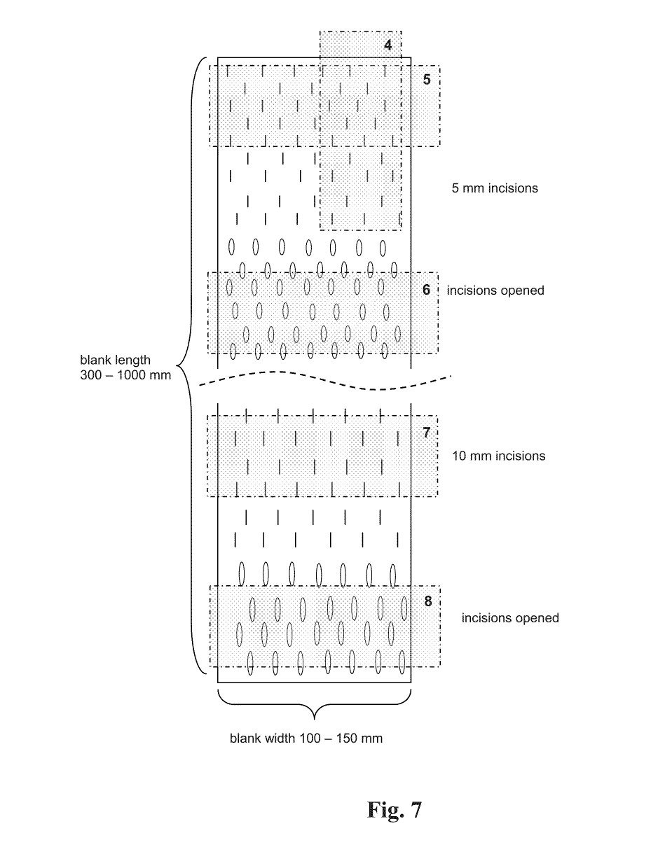

The effect of stretching to the pore area of incised composite samples is seen in FIG. 5. As will appear from the graph the stretching ratio linearly affects the pore area of samples. In non-stretched samples pore area is approximately 1% and in samples with 40% of stretching the pore area is approximately 8%.

### INDUSTRIAL APPLICABILITY

The present materials can be used in splints and circumferential casts. In sport appliances, such as grips for rackets in rackets sports, as well as in the above-mentioned foot-supporting applications, the capability of the material easily to be formed and exhibiting a degree of softness or elasticity is of particular use.

### CITATION LIST

### Patent literature

WO 2006/027763A2,

WO 2007/035875,

US 2008/0262400,

US 2012/0071590

WO 94/03211

1. A composite material in the shape of a three-dimensional object, comprising a first component formed by a polymer and a second component formed by a reinforcing material, wherein
said first component comprises a thermoplastic polymer selected from the group of biodegradable polyesters and mixtures thereof, and
said second component comprises particles of a woody material, having a smallest dimension greater than 0.1 mm,
said composite material further comprising
regions of elasticity formed by mechanical processing of the material to provide for objects having properties of flexibility or semi-rigidity in at least one dimension, and
said material being moldable and workable at temperatures below 70° C.

2. The composite material according to claim 1, further comprising perforations forming at least one region of flexibility.

3. The composite material according to claim 1, wherein the biodegradable polyester has a melting point below 70° C. and higher or equal to 55° C.

4. The composite material according to claim 1, wherein biodegradable polyester forms the matrix of the composite material.

5. The composite material according to claim 1, wherein the first component forms the matrix of the composite material, and the second component exhibits a microstructure which is discontinuous.

6. The composite material according to claim 1, wherein the biodegradable polyester is selected from the group of epsilon-caprolactone homopolymers, blends of epsilon-caprolactone homopolymers and other biodegradable thermoplastic homopolymers, with 5-99 wt % of an epsilon-caprolactone homopolymer and 1-95 wt % of a biodegradable thermoplastic polymer, and copolymers of epsilon-caprolactone homopolymer and any thermoplastic biodegradable polymer, with 5 to 99 wt % of repeating units derived from epsilon-caprolactone and 1 to 95 wt %, repeating units derived from other polymerizable material.

7. The composite material according to claim 1, further comprising a first polymer component having a melt flow index of 0.3-2.3 g/min (at 80° C.; 2.16 kg).

8. The composite material according to claim 1, wherein the three dimensional object comprises incisions, forming a region of flexibility.

9. The composite material according to claim 1, wherein the material is shaped into a planar object having a longitudinal and lateral axis and wherein the regions of elasticity are unidirectional either along the longitudinal or lateral axis.

10. The composite material according to claim 1, further comprising incisions having a width of 0.1 to 1 mm and a length of 4 to 20 mm.

11. The composite material according to claim 1, further comprising incisions made with a blade, the surface area of which incisions being on the blade ingoing side being 1 to 10 mm².

12. The composite material according to claim 1, further comprising incisions, the amount of the incisions/10 cm² being 20 to 100.

13. The composite material according to claim 1, further comprising linear incisions.

14. The composite material according to claim 1, further comprising a plurality of consecutive linear incisions.

15. The composite material according to claim 1, further comprising consecutive incisions arranged in a plurality of parallel lines.

16. The composite material according to claim 1, wherein the distance between two consecutive incisions in longitudinal direction is greater than 5 mm and less than 20 mm.

17. The composite material according to claim 1, wherein the space between each incision transversally to a next incision is greater than 10 mm and smaller than 25 mm.

18. The composite material according to claim 1, wherein the incisions are located such that they are kept closed in the direction of the three-dimensional object requiring maximal strength so as not to impair mechanical strength of the object in that direction.

19. The composite material according to claim 1, wherein the incisions are longitudinally directed such that they will therefore not be opened by the action of forces acting upon the object longitudinally.

20. The composite material according to claim 1, wherein the material is capable of yielding openings or apertures upon stretching of incisions.

21. The composite material according to claim 1, wherein the material exhibits, apertures having round, rectangular, square, diamond, hexagonal, oval, slot or ornamental shape.

22. The composite material according to claim 1, wherein the material exhibits in stretched condition a pore area of 2.5 to 30% of the total area.

23. The composite material according to claim 1, wherein the individual wood particles have at least two dimensions greater than 1 mm and one greater than 0.1 mm.

24. The composite material according to claim 1, wherein the wood particles are capable of being orientated and aligned in a melt flow of the thermoplastic polymer.

25. The composite material according to claim 1, wherein the wood particles comprise chips of hardwood, softwood or a combination thereof.

26. The composite material according to claim 1, said material being formable at a temperature of 50 to 70° C. and being rigid at a temperature of less than 50° C.

27. A method of producing a composite material in the shape of a three-dimensional object, comprising combining
a first component which is formed by a thermoplastic polymer selected from the group of biodegradable polyesters and mixtures thereof with
a second component which is formed by particles of a woody material, having a smallest dimension greater than 0.1 mm to produce a composite material,
shaping the material into a three dimensional object and, at an optional point of time,
introducing by mechanical processing of the object into the composite material regions of elasticity to provide the objects with properties of flexibility or semi-rigidity in at least one dimension
wherein the composite material comprises a first component formed by a polymer and a second component formed by a reinforcing material, wherein
said first component comprises a thermoplastic polymer selected from the group of biodegradable polyesters and mixtures thereof, and
said second component comprises particles of a woody material, having a smallest dimension greater than 0.1 mm,
said composite material further comprising
regions of elasticity formed by mechanical processing of the material to provide for objects having properties of flexibility or semi-rigidity in at least one dimension, and
said material being moldable and workable at temperature below 70° C.

28. The method according to claim 27, wherein perforations are formed into the composite material.

29. The method according to claim 27, wherein incisions are made with a blade, said incisions having a width of 0.1 to 1 mm and a length of 4 to 10 mm.

30. The method according to claim 27, wherein the size of the individual incisions are different on the opposite sides of the composite profile.

31. The method according to claim 27, wherein incisions are manufactured into the object with an incision device, or by water jet, or by laser cutting.

32. Splints and circumferential casts, comprising a composite material in the shape of a three-dimensional object, the composite material comprising a first component formed by a polymer and a second component formed by a reinforcing material, wherein
said first component comprises a thermoplastic polymer selected from the group of biodegradable polyesters and mixtures thereof, and
said second component comprises particles of a woody material, having a smallest dimension greater than 0.1 mm,
said composite material further comprising
regions of elasticity formed by mechanical processing of the material to provide for objects having properties of flexibility or semi-rigidity in at least one dimension, and
said material being moldable and workable at temperature below 70° C.
